(19) United States
(12) Patent Application Publication
(10) Pub. No.: US 2025/0111940 A1
(43) Pub. Date: Apr. 3, 2025

(54) PERSONAL HEALTH ASSISTANT CHATBOT: INTEGRATED PATIENT INTAKE, DATA STORAGE AND PERSONALIZED MEDICAL RECOMMENDATIONS SYSTEMS AND METHODS

(71) Applicant: Michael Dent, Naples, FL (US)

(72) Inventor: Michael Dent, Naples, FL (US)

(21) Appl. No.: 18/900,727

(22) Filed: Sep. 28, 2024

Related U.S. Application Data
(60) Provisional application No. 63/541,016, filed on Sep. 28, 2023.

(51) Int. Cl. G16H 40/67; G06T 13/40; G16H 10/60; G16H 50/20

(52) U.S. Cl. CPC G16H 40/67; G06T 13/40; G16H 10/60; G16H 50/20

(57) ABSTRACT

A personal health assistant (PHA) chatbot system employing large language model (LLM) to a) receive a patient query; b) prompt for a patient identifier and a personal medical inquiry record; c) create a prompt record based on a patient-specific datum stored in secure medical profile; d) creating a dynamic prompt characterized by the prompt record; and e) updating an avatar-based interface based on the dynamic prompt. Patient-specific data (e.g., medical history, medications, and allergies) associated with the patient identifier may be updated with a patient profile change retrieved from an external data source(s) across a network interface (e.g., an electronic medical record (EMR)). The system matches common health issue types (e.g., location, medications, genetics, medical history, activities, environmental exposures), as well as other pertinent variables (e.g., language, culture, age, gender, and/or ethnicity) to the patient-specific data to deliver a medical recommendation and/or a personalized health advice through a user-tailored avatar.

### CROSS-REFERENCE TO RELATED APPLICATIONS

This application claims the benefit under 35 U.S.C. § 119 (e) of U.S. Provisional Patent Application Ser. No. 63/541,016 titled PERSONAL HEALTH ASSISTANT CHATBOT: INTEGRATED PATIENT INTAKE, DATA STORAGE, AND PERSONALIZED MEDICAL RECOMMENDATIONS SYSTEMS AND METHODS filed on Sep. 28, 2023, the entire contents of which are incorporated herein by reference.

### FIELD OF THE INVENTION

The present invention relates broadly to human conversation simulation technology and advanced patient data management. Specifically, this invention encompasses computer-implemented devices, systems, and associated methods that utilize natural language processing (NLP) powered by a large language model (LLM) algorithm for patient assessment and diagnosis in a medical setting. Beyond intake, the system may provide storage, organization, and query of individual patient data; advantageously facilitating the provision of personalized recommendations for routine health screenings and exams, as well as tailored medical advice based on a patient's unique medical history.

### BACKGROUND OF THE INVENTION

The practice of medical diagnosis has benefited from recent advancements in computing designed to improve the efficiency and effectiveness of patient-facing medical professionals. One disruptive technology that promises to augment or at least partially replace humans in the medical diagnosis role is natural language processing (NLP). NLP is a branch of artificial intelligence (AI) that enables computers to understand and process human language.

One goal for employment of NLP in a medical setting is to understand patient questions and to respond with health-enhancing guidance in a conversational format. The more natural the dialogue between patient and NLP-enabled computer, the better the information intake gleaned from patients and, ultimately, the more accurate and useful the automated diagnoses proposed based on that information. For example, because of the post-capture structure imposed upon the natural language input, human-in-the-loop medical professionals may be better equipped with coherent medical records that may be quickly searched and analyzed to support a review-ready diagnosis. Such automation of the diagnosis process may advantageously allow medical professionals to focus more time on patient care rather than on mundane intake and recording tasks, thereby reducing the cost of medical diagnosis and increasing accessibility to “live” medical professionals for those tasks that uniquely depend on human judgment.

Despite the potential for improving patient care, use of NLP for medical diagnosis poses several challenges, such as the following:
1) Data accuracy: NLP-based automation must be trained on accurate, accessible, and up-to-date data, including patient medical histories, current medications, and other relevant information (e.g., environmental variables).
2) Data interpretation: To achieve desired effectiveness, NLP-based automation must be able to accurately interpret the data it is given. Such systems must be able to recognize patterns in the input data and to accurately predict based on those patterns.
3) Data security: Patient data input for medical diagnosis purposes must be kept confidential and saved securely. Malicious access for the purpose of manipulating or destroying patient data must be monitored and prevented.

The advent of large language model (LLM) algorithms such as generative pre-trained transformer (GPT) has opened new frontiers in conversational AI, enabling computers to understand and generate human-like text. In a medical context, these models can automate and augment the diagnostic process by capturing detailed patient data through natural conversation, providing personalized guidance, and identifying patterns across large datasets of patient information.

However, existing applications of LLMs in healthcare face several challenges:
(4) Contextual Understanding and Relevance: Known systems do not fully utilize LLM's ability to dynamically inject relevant patient data (e.g., medical history, medications, allergies) into responses, often resulting in less precise or potentially irrelevant medical guidance.
(5) Data Integration and Pattern Recognition: Many solutions fail to integrate LLMs effectively with existing healthcare data systems, such as electronic medical records (EMRs), to identify common issues across patients, such as location-specific conditions, medication side effects, genetic predispositions, and environmental exposures.
(6) Patient Onboarding and Profile Creation: Known systems often lack a user-friendly method for patients to onboard and provide their comprehensive medical history in a secure manner.
(7) Multi-language and Cultural Adaptation: Known systems often lack the ability to seamlessly support multiple languages and adapt to the cultural and personal preferences of diverse patient populations.
(8) Security and Privacy: The use of LLM algorithms requires robust security measures to protect sensitive patient data and ensure compliance with healthcare regulations.

Accordingly, a need exists for an enhanced system that leverages LLM algorithms for improved data capture, contextual understanding, real-time prompt injection, pattern recognition across patient users, multi-language and culturally adaptive responses, interactive patient onboarding, and the delivery of standard medical guidance, while ensuring robust security and compliance.

This background information is provided to reveal information believed by the applicant to be of possible relevance to the present invention. No admission is necessarily intended, nor should be construed, that any of the preceding information constitutes prior art against the present invention.

### SUMMARY OF THE INVENTION

With the above in mind, embodiments of the present invention are related to an advanced patient data management system that may utilize a large language model (LLM) algorithm such as a generative pre-trained transformer (GPT) platform for human conversation simulation in medical settings. More specifically, the present invention may comprise a computer-implemented system employing natural language processing (NLP) powered by an LLM to enhance patient assessment, diagnosis, personalized medical care, and population-level health insights. The system may be configured to advantageously feature dynamic prompt injection capabilities, multi-language support, customizable virtual physician interfaces, and an interactive onboarding process for collecting patient medical history.

For example, and without limitation, certain embodiments of the present invention may comprise medical diagnosis automation in the form of a personal health assistant (PHA) chatbot comprising an avatar-based, multilingual, and/or customizable interface designed to interact with patients in real-time by gathering medical history, creating a comprehensive medical profile, and delivering personalized healthcare guidance.

More specifically, in a computer-implemented method aspect of the present invention, personal health assistant (PHA) chatbot operations may be performed using a large language model (LLM) platform. The method may comprise the steps of a) receiving a patient query of one of an onboarding command type and a recommendation command type; 2) upon detection of an onboarding command, prompting for and receiving a patient identifier, at least one account setup record, and at least one personal healthcare record, and; 3) storing the personal healthcare record(s) to a secure medical profile associated with the patient identifier and characterized by the account setup record(s). Upon detection of a recommendation command, the method may comprise the steps of 4) prompting for and receiving a patient identifier and a personal medical inquiry record; 5) creating a prompt record based on at least one patient-specific datum of the secure medical profile associated with the patient identifier; 6) creating a dynamic prompt characterized by the prompt record; and 7) updating an avatar-based interface based on the dynamic prompt.

The computer-implemented method may further comprise the steps of 8) updating the patient-specific data (e.g., medical history, medications, and allergies) of the secure medical profile associated with the patient identifier with a patient profile change that may be 9) retrieved from an external data source across a network interface (e.g., an electronic medical record (EMR)). The computer-implemented method may further comprise the steps of 10) matching a common health issue type (e.g., location, medications, genetics, medical history, activities, environmental exposures) to the patient-specific data of the secure medical profile associated with the patient identifier; and 11) creating the prompt record based at least in part on the common health issue type. Alternatively, or in addition, the prompt record may be 12) created based on a language type, a culture type, an age type, a gender type, and/or an ethnicity type associated with the patient identifier. The computer-implemented method may further comprise the step of 13) creating the prompt record based a medical recommendation and/or personalized health advice.

These and other objects, features, and advantages of the present invention will become more readily apparent from the attached drawings and the detailed description of the preferred embodiments, which follow.

### BRIEF DESCRIPTION OF THE DRAWINGS

The preferred embodiments of the invention will hereinafter be described in conjunction with the appended drawings provided to illustrate and not to limit the invention, where like designations denote like elements, and in which:

FIG. 1 is a function diagram of a personal health assistant (PHA) chatbot system according to an embodiment of the present invention;

FIG. 2 is a schematic block diagram of a personal health assistant (PHA) chatbot system according to an embodiment of the present invention;

FIG. 3A is a first exemplary component diagram of the personal health assistant (PHA) chatbot system of FIG. 2;

FIG. 3B is a second exemplary component diagram of the personal health assistant (PHA) chatbot system of FIG. 2;

FIG. 3C is a third exemplary component diagram of the personal health assistant (PHA) chatbot system of FIG. 2;

FIG. 3D is a fourth exemplary component diagram of the personal health assistant (PHA) chatbot system of FIG. 2; and

FIG. 4 is a sequence diagram of an exemplary method aspect of the personal health assistant (PHA) chatbot system of FIG. 2.

Like reference numerals refer to like parts throughout the several views of the drawings.

### DETAILED DESCRIPTION OF THE INVENTION

The present invention will now be described more fully hereinafter with reference to the accompanying drawings, in which preferred embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art.

Although the following detailed description contains many specifics for the purposes of illustration, anyone of ordinary skill in the art will appreciate that many variations and alterations to the following details are within the scope of the invention. Accordingly, the following embodiments of the invention are set forth without any loss of generality to, and without imposing limitations upon, the claimed invention.

As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims.

Furthermore, in this detailed description, a person skilled in the art should note that quantitative qualifying terms such as “generally,” “substantially,” “mostly,” and other terms are used, in general, to mean that the referred to object, characteristic, or quality constitutes a majority of the subject of the reference. The meaning of any of these terms is dependent upon the context within which it is used, and the meaning may be expressly modified.

In general, embodiments of the present invention may utilize a computer-operated LLM platform (e.g., GPT-based) to power a personal health assistant (PHA) chatbot system designed to interact conversationally with a system user (e.g., medical patient). The chatbot system may leverage the LLM platform's ability to understand and generate human-like text to facilitate dynamic patient interactions and personalized medical recommendations. Additionally, the chatbot system may employ prompt injection techniques, pattern recognition capabilities, multi-language support, customizable virtual physician interfaces, and a secure onboarding process for collecting patient medical history.

Components and functions of certain embodiments of the present invention may include the following:
LLM-Powered Conversational User Interface: An avatar-driven user interface that may engage patients in natural dialogue, capturing detailed medical histories, symptoms, and lifestyle information. The LLM may dynamically adjust its responses based on real-time inputs, ensuring accurate data capture;
Prompt Injection Technology: When a patient asks a specific question-such as an inquiry about a symptom (e.g., a rash)—the system may inject relevant patient data, including current medical history, medications, and known allergies, into the LLM prompt. This process may allow the system to formulate the most likely cause of the symptom, providing a highly accurate and personalized response. For example, in a scenario where the patient presents symptoms such as dizziness, the LLM, through prompt injection, may integrate the patient's pre-existing conditions like hypertension and current medications, such as beta-blockers, into the query. This capability may allow for a contextually relevant diagnosis and the formulation of a tailored follow-up question or health suggestion;
Data Integration and Profile Management: Seamless integration with EMR systems and external data sources may allow the system to compile a comprehensive patient profile. The LLM platform's capabilities may enable it to query and interpret this data for more precise medical recommendation. The system's LLM may be fine-tuned across various healthcare specializations, enabling the chatbot to provide domain-specific guidance. By dynamically switching between these fine-tuned models, the system may efficiently scale across different healthcare settings without sacrificing accuracy;
AI-Based Decision Support System (DSS): The system may serve as an AI-based decision support system (DSS), providing healthcare professionals with actionable insights derived from aggregated patient data. This allows the system to assist in identifying high-risk patients, optimizing treatment plans, and recommending personalized therapies, making the system not only a patient-facing tool but also a valuable resource for clinicians.
Contextual Awareness for Environmental and Temporal Factors: The system also incorporates contextual factors, such as environmental conditions (e.g., air quality, weather) and temporal patterns (e.g., seasonal health risks), to enhance the accuracy of medical assessments. By dynamically adjusting recommendations based on these variables, the system may provide a more holistic view of patient health.
Patient Onboarding and Profile Creation: The system may allow patients to interact with the AI-powered interface through a user-friendly onboarding process. Patients can verbally communicate their medical history, medications, allergies, and other personal healthcare information, which the system may securely store in a patient's system-managed health profile (referred to hereinafter as a HealthLynked network profile). This process may ensure accurate data collection and may enhance patient engagement by creating a comprehensive and secure healthcare profile. The chatbot not only may facilitate patient onboarding with real-time conversational interaction but also may monitor patient data to suggest timely follow-up actions, such as routine screenings or medication adjustments, ensuring continuous personalized care;
Pattern Recognition Across Patient Users: The system may analyze aggregated data from multiple patient users to identify common issues related to medical conditions. This analysis may include examining factors such as geographic location, medication use, genetic predispositions, medical history, daily activities, and environmental exposures. The system may identify trends and correlations that may indicate emerging public health concerns and/or patient-specific patterns;
Incorporation of Real-World Evidence (RWE): The system integrates real-world evidence (RWE) from diverse patient populations to continuously refine its diagnostic algorithms and medical recommendations. This feedback loop allows for real-time adjustments based on patient outcomes, improving both short- and long-term decision-making capabilities;
Multi-language and Culturally Adaptive Responses: The system may be configured to understand and respond in a language chosen by the patient. The system may employ the LLM platform's capabilities to provide culturally appropriate medical guidance, ensuring accessibility and relevance to diverse patient populations. To ensure equitable care and unbiased recommendations, the system may automatically adjust for cultural, linguistic, and demographic differences by incorporating AI models trained on diverse datasets. Doing so may mitigate the risk of biased medical advice, ensuring accuracy for patients from various backgrounds.
Adaptation to Emerging Diseases and Public Health Crises: The system is equipped to adapt to emerging public health crises, such as pandemics, by analyzing real-time patient data and identifying early warning signs of new health threats. This proactive feature may allow healthcare providers to respond more quickly to emerging trends and implement preventive measures on a large scale.
Customizable Virtual Physician Interface: The system may create and present a virtual physician avatar that may be customized according to the patient's preferences. Patients can choose the gender and ethnicity of the virtual physician, creating a more personalized and comfortable interaction experience;
Standard Medical Guidance: The system may automatically provide patients with standard medical guidance based on routine screening and vaccine guidelines, taking into account the patient's age, gender, medical history, and known risk factors. This guidance may be continually updated to reflect the latest medical standards and recommendations
AI-Driven Proactive Health Management: The system leverages AI-driven analytics to continuously monitor patients' health data and provide proactive suggestions for preventive care. This includes personalized reminders for routine health checks, vaccines, and lifestyle adjustments based on predictive health risk models.
Personalized Health Guidance: The LLM platform may generate tailored recommendations by analyzing patient data, recognizing patterns, and understanding context-specific medical concerns. This guidance may include routine screenings, preventive care measures, or follow-up actions, all delivered in a manner that may mimic natural human conversation; and/or
Secure and Compliant Data Handling: The system may employ robust security measures, including encryption and multi-factor authentication, to protect patient data. The system may be designed to comply with relevant healthcare regulations such as the Health Insurance Portability and Accountability Act (e.g., HIPAA) to ensure legal and ethical use of data. The system may incorporate robust security measures, including end-to-end encryption with AES-256 during patient data storage and transmission. Multi-factor authentication (MFA) may be enforced for access control, ensuring compliance with HIPAA and General Data Protection Regulation (GDPR) standards for handling sensitive health data.

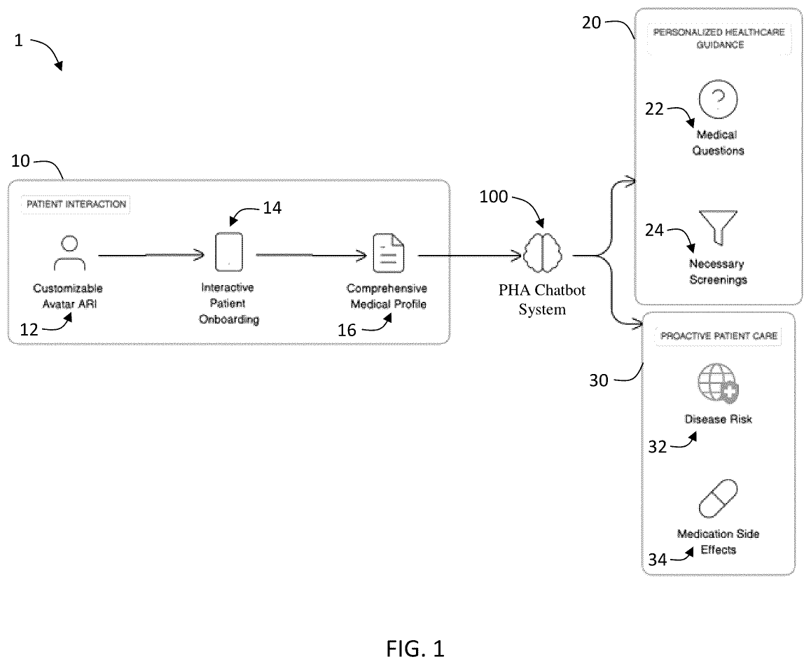

Referring now to the function diagram 1 of FIG. 1, a personal health assistant (PHA) chatbot system 100 is illustrated in accordance with an exemplary embodiment of the present invention. Those skilled in the art will understand that the principles of the present disclosure may be implemented on or in data communication with any type of suitably arranged device or system configured to simulate human conversation/dialogue.

As shown in FIG. 1, certain embodiments of a personal health assistant (PHA) chatbot system 100 of the present invention may comprise a computing system configured to prompt and interpret human language as spoken and written by a medical patient. For example, and without limitation, this patient interaction 10 may allow employed computing resources to understand human language using artificial intelligence (AI) techniques. More specifically, patient interaction 10 may comprise presentation of a customizable avatar 12 that may lead an automated, real-time dialogue with a user to conduct interactive patient onboarding 14. Medical and identifying information then may be used by the PHA chatbot system 100 to build a comprehensive medical profile 16 for the user. The PHA chatbot system 100 may perform automated analysis of the comprehensive medical profile 16 to determine for the user personalized healthcare guidance 20 and/or proactive patient care 30 suggestions. Personalized healthcare guidance 20 may be presented by the PHA chatbot system 100 in the form of medical questions 22 pertinent to the patient and/or necessary screenings 24 tailored to the querying patient. Proactive patient care 30 may be presented by the PHA chatbot system 100 in the form of reports (either textual and/or audible) on disease risk 32 and/or medication side effects 34 tailored to the querying patient.

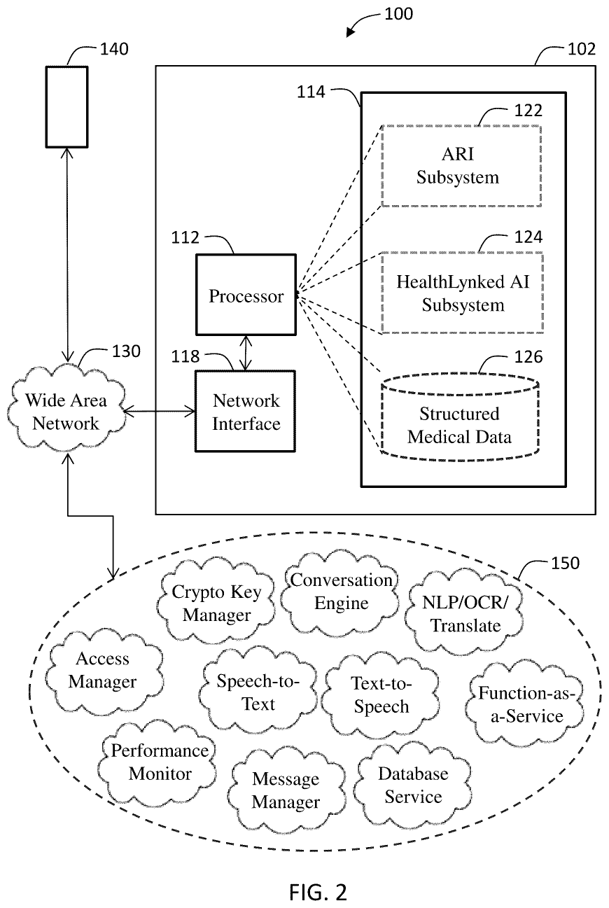

Referring now to the schematic block diagram of FIG. 2, certain embodiments of a PHA chatbot system 100 of the present invention may comprise a processor 112 (also referred to herein as a “microprocessor” or “central processing unit (CPU)) that may be operable to accept and execute computerized instructions, and also a data store 114 that may store data and instructions used by the processor 112. The processor 112 may be positioned in data communication with some number of external devices and may be configured to direct input from such external devices to the data store 114 for storage and subsequent retrieval. For example, and without limitation, the processor 112 may be configured in data communication with downstream networking resources (e.g., a wide area network (WAN) 130) via a network interface 118 (e.g., internet connection port). Using such data interfaces, the processor 112 may be configured to direct input received from components of the WAN 130 (e.g., a web resource or a smartphone 140) to the data store 114 for storage and manipulation. Similarly, the processor 112 may be configured to retrieve data from the data store 114 to be forwarded as output to various components of the WAN 130 (e.g., web resource or the smartphone 140).

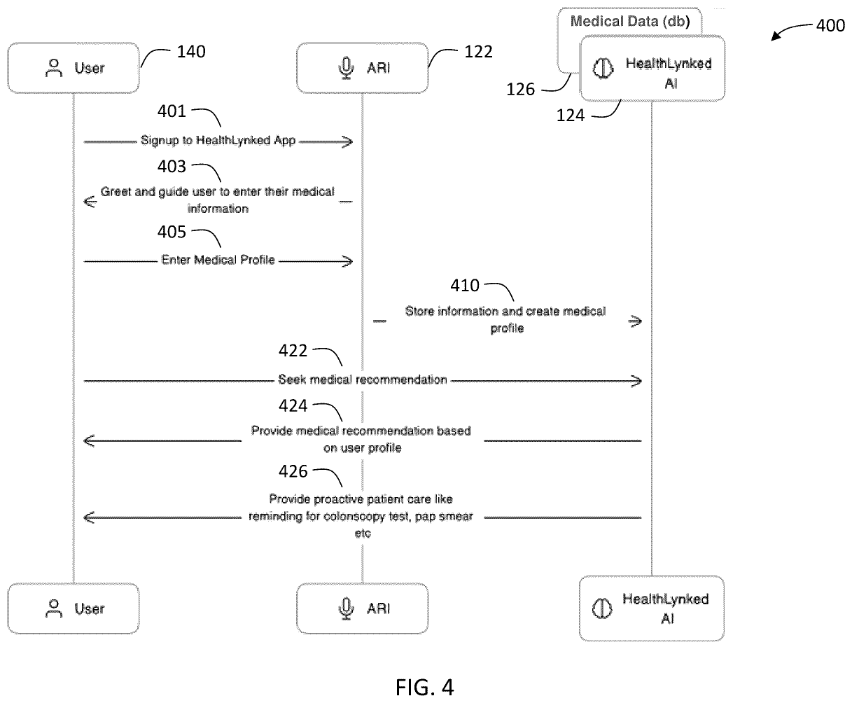

For example, and without limitation, the computerized instructions of the PHA chatbot system 100 may be configured to implement an Automated Real-time Interface (ARI) Subsystem 122, a HealthLynked AI Subsystem 124 (also referred to as a Query Engine), and/or a database of Structured Medical Data 126 that may be stored in the data store 114 and retrieved by the processor 112 for execution. The ARI Subsystem 122 may be operable to implement functionality as described hereinbelow (e.g., for FIG. 4) for a customizable avatar as an interface for use by a medical patient. The HeathLynked AI Subsystem 124 may be operable to implement functionality as described hereinbelow (e.g., for FIG. 4) for intake data manipulation and medical diagnosis/proactive patient care development. The database 126 may be operable to provide functionality as described hereinbelow (e.g., for FIG. 4) for storage and retrieval of Structured Medical Data for use by the present invention and/or by downstream medical care delivery systems and/or consumers (e.g., medical professionals).

A person of skill in the art will immediately recognize that an advantageous implementation of the PHA chatbot system 100 may employ any or all of an ARI Subsystem 122, HealthLynked AI Subsystem 124, and/or database of Structured Medical Data 126 collocated upon a single host computing device or distributed among two or more host computing devices. For example, and without limitation, the various components of the PHA chatbot system 100 may be implemented onboard an application host/server 102. Alternatively, or in addition, any or all components of the PHA chatbot system 100 may employ cloud services 150 (e.g., applications and/or storage) accessible from distributed computing resources support by data centers providing such services via wide area network 130.

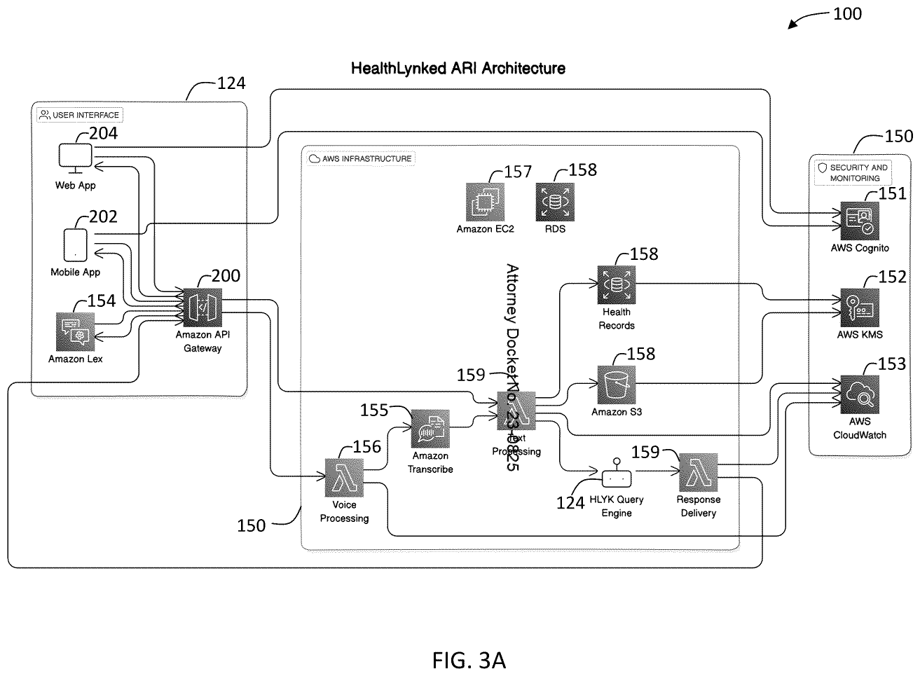

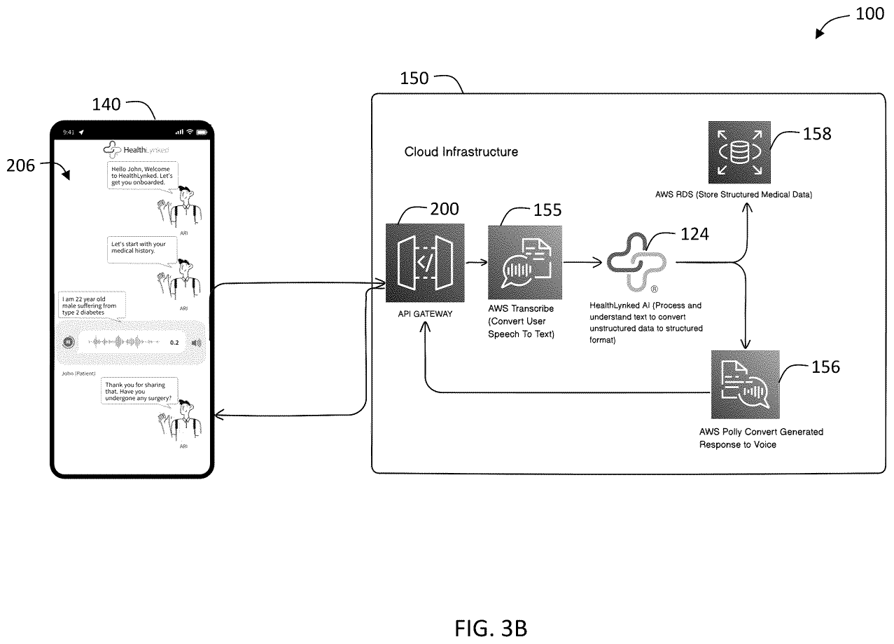

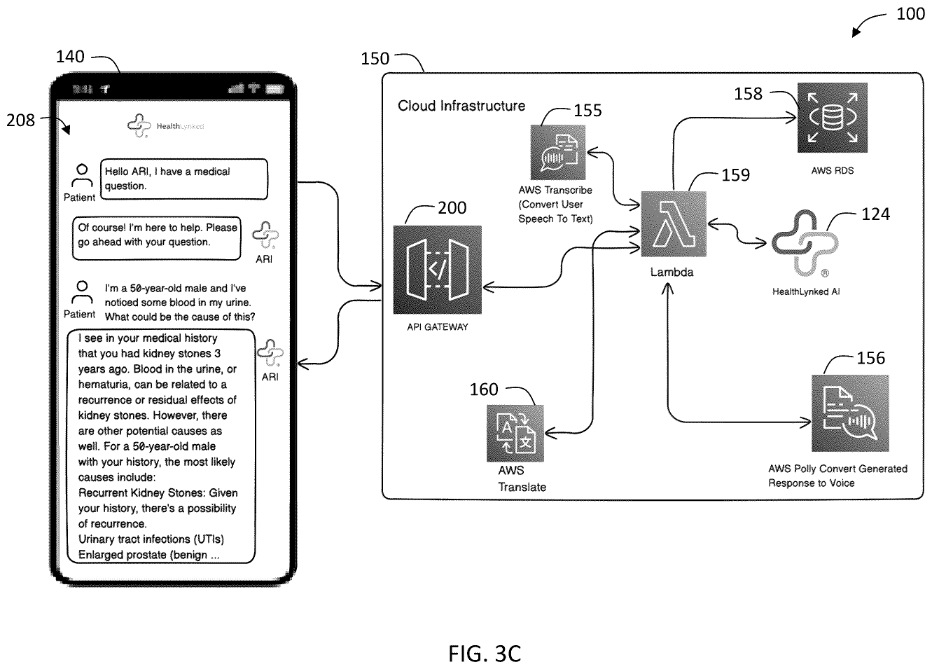

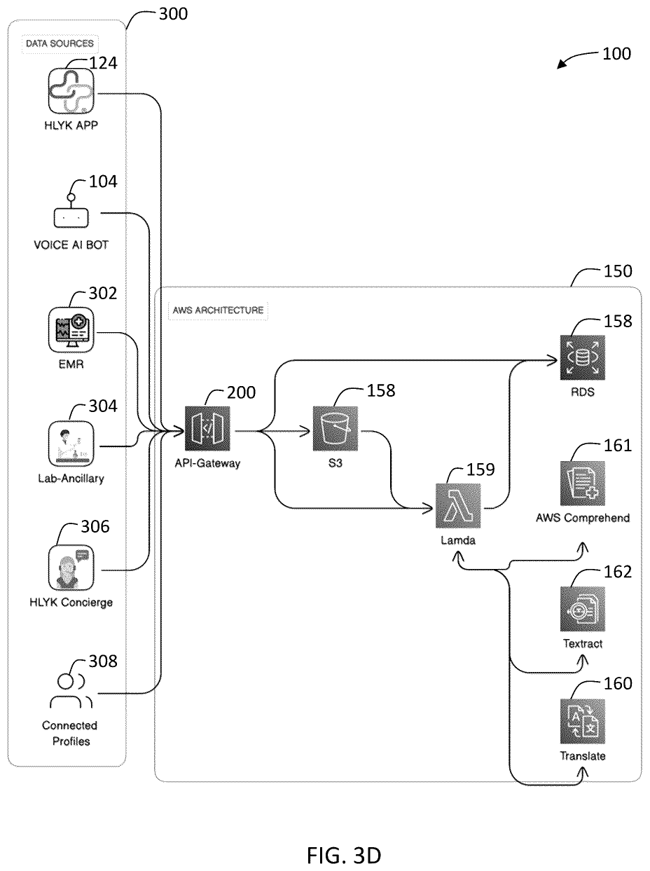

Still referring to FIG. 2, and referring additionally to the exemplary component diagram of FIG. 3A, the following are examples of cloud-based web services 150 that may be employed in certain embodiments of the present invention (Note: Although certain on-demand cloud computing solutions proprietary to Amazon Web Services, Inc. are mentioned hereinbelow, a person of skill in the art will immediately recognize that any similarly configured web services may be employed in keeping with the present invention design to deliver the advantageous functionality as described herein):

Access manager 151 (e.g., Amazon® Cognito)—web services for addition of user sign-up and authentication to mobile and web apps, and for managing identities and access controls; provides security credentials to access backend resources of the supported app.

Crypto key manager 152 (e.g., Amazon® Key Management Service (KMS))—web service for creation, management, and control of cryptographic keys for use in a supported app; provides data protection, encryption/decryption, and digital signature/verification.

Performance Monitor 153 (e.g., Amazon® CloudWatch)—web service for visualizing and monitoring system-wide operational health across various cloud resources and applications; automated notifications flag performance issues for mediation.

Conversation engine 154 (e.g., Amazon® Lex)—web service for building chatbots with conversational artificial intelligence (AI); employs natural language models to design, build, test, and deploy conversational interfaces in a supported app.

Speech-to-text 155 (e.g., Amazon® Transcribe)—web service for automatic speech recognition (ASR) for use within a supported app; document conversations into system-friendly data records for subsequent analysis.

Text-to-speech 156 (e.g., Amazon® Polly)—web service for synthesizing textual data into natural-sounding human speech in a desired language(s); delivers conversational user experiences within a supported app.

Cloud Compute Capacity 157 (e.g., Amazon® Elastic Compute Cloud (EC2))—web service for accessing on-demand computing infrastructure; provides processor, storage, networking, and/or operating system platform(s) on the cloud.

Database service 158 (e.g., Amazon® Relational Database Service (RDS))—web service supporting an array of relational database engines; provides tools to perform database management tasks such as data migration, backup, recovery and patching.

Function-as-a-Service 159 (e.g., Amazon® Lambda)—web service delivering serverless, event-driven execution of code for a supported app or backend service; combines with other web services (e.g., Amazon® Transcribe for voice processing; Amazon® Polly for text processing; response delivery) to create secure, stable, and scalable online experiences.

The ARI Subsystem 122 may implement speech recognition using one or more known language models, for example, and without limitation, a large language model (LLM) algorithm such as generative pre-trained transformer (GPT). A person of skill in the art will immediately recognize that known alternative LLMs designed to generate natural language texts on various topics and tasks may be employed to implement the functions described hereinbelow for the ARI Subsystem 122.

Still referring to FIG. 2, and referring additionally to the exemplary component diagram of FIG. 3B, the core infrastructure of the illustrated embodiment of the PHA chatbot system 100 may be comprised solely of cloud-based web services 150, similar to the exemplary component diagram of FIG. 3A.

As illustrated in both FIGS. 3A and 3B, an application programing interface (API) manager (e.g., Amazon® API Gateway 200) may operate an avatar interface displayed as a mobile app 202 or a web app 204 (e.g., on a smartphone 140), for example, and without limitation, to support a patient onboarding use case (as illustrated in example display 206). User speech captured by the API Gateway 200 may be converted to text using AWS Transcribe 155. Such text may be processed by the HealthLynked API 124 (e.g., as implemented in Amazon Lambda 159) to convert unstructured data to structured format for subsequent storage to the AWS RDS 158. As needed, the HealthLynked API 124 may create patient-specific guidance that may be converted to voice by an AWS Polly 156 before being returned by the HealthLynked AI Subsystem 124 to the API Gateway 200 for communication in an audible (i.e., “spoken”) and/or textual (e.g., “written”) form to the user through the avatar interface presented by the mobile app 202 and/or the web app 204.

Still referring to FIG. 2, and referring additionally to the exemplary component diagram of FIG. 3C, the illustrated embodiment of the PHA chatbot system 100 again may be comprised solely of cloud-based web services 150, for example, and without limitation, including the following:

Multilingual Translation 160 (e.g., Amazon® Translate)—web service that performs multilingual sentiment analysis of social media content; provides on-demand translation of user-generated content and adds the real-time translation for communication applications.

As illustrated in FIG. 2, an avatar interface displayed as a mobile app 202 on a smartphone 140 may be operated by an API Gateway 200, for example, and without limitation, to support a patient guidance and referral use case (as illustrated 208). Logic flow similar to that described above for FIG. 3B may be followed for this use case. Specifically, user speech captured by the API Gateway 200 may be converted to text using AWS Transcribe 155. Such text may be processed by the HealthLynked API 124 (e.g., as implemented in Amazon Lambda 159) to convert unstructured data to structured format for subsequent storage to the AWS RDS 158. As needed, the HealthLynked API 124 may create patient-specific guidance that may be converted to voice by an AWS Polly 156 (and optionally translated into the patient's preferred language by an AWS Translate 160) before being returned by the HealthLynked AI Subsystem 124 to the API Gateway 200 for communication in an audible (i.e., “spoken”) and/or textual (e.g., “written”) form to the user of the smartphone 140 through the avatar interface (e.g., mobile app 202).

In certain embodiments of the present invention, patient data aggregation from a variety of input sources may be supported. Still referring to FIG. 2, and referring additionally to the exemplary component diagram of FIG. 3D, the following are examples of cloud-based web services 150 that may be employed to implement patient data aggregation:

Database service 158 (e.g., Amazon® Simple Storage Service (S3))—web service(s) supporting object storage configured for scalable and secure data storage and retrieval from a variety of data sources, such as data takes, cloud-native applications, and mobile apps; includes capabilities such as appending metadata tags, enforcing data access controls, and running data analytics.

Natural Language Processing (NLP) 161 (e.g., Amazon® Comprehend)—web service that extracts insights about the content of documents; employs machine learning to recognize entities, key phrases, language, sentiments, and other common elements in a document of interest.

Smart Optical Character Recognition (OCR) 162 (e.g., Amazon® Textract)—web service that reads and extracts text, handwriting, and data from scanned documents; employs machine learning to automatically identify, understand, and extract data from forms and tables.

So configured, the PHA chatbot system 100 may gather and aggregate input from a variety of sources to build a comprehensive patient profile, ensuring that a broad range of relevant health information is integrated and accessible. Data entry sources 300 include not only the HealthLynked AI Subsystem 124 and the related user interface (e.g., avatar displayed on a smartphone 140 by the ARI Subsystem 122); but also additional input sources managed by the common API Gateway 200. For example, and without limitation, the following relevant health data sources may provide data input for aggregation by the PHA chatbot system 100:

Patient Self-Input: In this automated method, patients directly input their data into the HealthLynked AI Subsystem 124 from a customer-facing device (e.g., smartphone 140). The type of data input includes, but is not limited to, patient medical history, current medications, allergies, surgical history, and symptoms currently being experienced.

Voice Conversion to Data: In this automated method, NLP is leveraged to allow patients to verbally communicate their health details. An interface may be presented (e.g., on a smartphone 104) to visually represent a doctor, enhancing the user experience by making patients feel as though they are conversing with an actual physician. The AI then translates this voice data into structured digital information. The type of data input includes any information that the patient might verbally provide, such as medical history or symptoms.

Electronic Medical Record (EMR) Systems: In this automated method, Application Programming Interfaces (APIs) are employed to pull relevant patient data from various EMR systems 302 (e.g., digitized paper medical charts). The type of data input may include previous diagnoses, treatment plans, medical notes, and other critical information that typically resides in an EMR.

Labs and Ancillary Services: In this automated method, laboratory test results and data from other ancillary services 304 may be integrated directly into the patient profile. The type of data input may include blood test results, imaging reports, and pathology findings that may provide a more in-depth understanding of a patient's current health status.

Telephone Onboarding with Human Representatives: In this semi-automated method, patients may telephone and share their medical data with live representatives (e.g., HealthLynked Concierge 306) who may input the provided data into the PHA chatbot system 100. The type of data input may be any information that a patient feels necessary to share or that the representative deems important to capture during the telephone call.

Connected Profiles: In this semi-automated method, data from family members of a patient may be linked or added to the patient profile. This type of data input primarily focuses on family medical history, which may be pivotal in assessing risks for certain diseases. For instance, if connected profiles 308 of a family member show a history of a specific type of cancer or heart disease, the patient might be at a higher risk for the same condition(s).

A person of skill in the art will immediately recognize that additional data input means and/or interfaces may be employed to implement the functions described herein for the PHA chatbot system 100. Such breadth of data gathering and subsequent aggregation advantageously offers a holistic and comprehensive approach to creating patient profiles, ensuring that healthcare providers are provided a well-rounded view of a patient's health and facilitating development of more informed medical decisions.

The present disclosure contemplates the use of computer instructions and/or systems configurations that may perform any or all of the operations involved in human conversation simulation. The disclosure of computer instructions that include ARI Subsystem 122 instructions, HealthLynked AI Subsystem 124 instructions, and/or data manager 126 instructions is not meant to be limiting in any way. Those skilled in the art will readily appreciate that stored computer instructions and/or systems configurations may be configured in any way while still accomplishing the many goals, features and advantages according to the present disclosure.

Referring now to the sequence diagram of FIG. 4, and still referring to FIGS. 1 and 2, a method aspect 400 of the PHA chatbot system 100 of the present invention will now be described in detail. From the start at Step 401, a user 140 (e.g., computing device operated by a medical patient) may interact with the ARI Subsystem 122 to create a user account (e.g., signup 401 by creating identification/credential information). Upon account creation, the ARI Subsystem 122 may present a customizable avatar to greet and guide 403 the user 140 to enter pertinent medical information (e.g., in the user's language of choice). The user 140 may engage in a human language dialogue with the avatar of the ARI Subsystem 122 to capture/enter 405 medical profile information. At step 410, the ARI Subsystem 122 may pass the patient information gathered through the conversational dialogue to the HealthLynked AI Subsystem 124 for structuring of the user's medical profile and storage of this personalized information to the system database 126. Subsequent on-demand queries by the user 140 of the avatar of the ARI Subsystem 122 may seek medical recommendations, which may be processed by the HealthLynked AI Subsystem 124 in data communication with the database holding the Structured Medical Data 126 for the user/patient 140. Patient-specific guidance may be returned by the HealthLynked AI Subystem 124 and communicated in spoken and/or written form to the user 140 through the ARI Subsystem 122. For example, and without limitation, the patient-specific guidance may comprise a medical recommendation(s) 424 based on the medical profile of the user 140, and/or proactive patient care 426 tailored to the user 140 (e.g., reminders for needed tests such as a colonoscopy or a pap smear).

In certain embodiments of the present invention, Steps 403 and 405 of the method 400 of FIG. 4 may comprise Patient Information Intake, to include collecting user's 140 personal and medical information such as the following:
A) basic demographic information (name, age, gender),
B) detailed medical history (past diagnoses, surgeries, allergies, etc.),
C) family medical history (known instances of diseases in the user's family),
D) lifestyle information (smoking, alcohol, exercise, diet),
E) current symptoms (if any), and
F) current medications.

In certain embodiments of the present invention, Step 410 of the method 400 of FIG. 4 may comprise Information Aggregation and Organization, to include processing and organizing the collected information such as the following:
G) creating a health profile for the user based on the input data,
H) classifying data into different categories like allergies, past diagnoses, family history, etc., and
I) making data searchable for future reference and updates.

Optional functionality for augmenting health profile information may include user input and management of information about current healthcare providers and hospitals, store contact information, and links to appointments, procedures, test results, and/or insurance information related to specific providers.

In certain embodiments of the present invention, Steps 422, 424 and 426 of the method 400 of FIG. 4 may comprise Artificial Intelligence Processing, to include the utilization of AI algorithms for effective decision-making, such as the following:
J) suggesting routine screenings based on the user's demographic details, and communicating recommendations regarding the same,
K) suggesting personalized screenings based on a user's specific medical history, and communicating recommendations regarding the same, and
L) generating unique prompt injections based on the user's profile and their symptoms or medical queries to provide more personalized and contextually accurate medical advice.

The AI Processing capability of the present invention may include Interactive Medical Query Handling, as a means of providing an interface for the user to ask medical questions based on their symptoms. In certain embodiments, a chat interface is presented for inputting of user queries. The AI analyzes the query in the context of the user's medical history and provides personalized advice.

The comprehensive computer-implemented framework described hereinabove outlines how ARI acts as a personal health assistant, providing information and advice based on a user's personal medical history and current symptoms, streamlining healthcare management, and enhancing the user's healthcare experience. In keeping with medical profession ethics, the automation described herein may be programmed to routinely encourage users to seek professional medical advice when necessary and not to presume the PHA chatbot replaces the role of healthcare professionals.

In some embodiments, the method or methods described above may be executed or carried out by a computing system including a tangible computer-readable storage medium, also described herein as a storage machine, that holds machine-readable instructions executable by a logic machine (i.e., a processor or programmable control device) to provide, implement, perform, and/or enact the above-described methods, processes and/or tasks. When such methods and processes are implemented, the state of the storage machine may be changed to hold different data. For example, the storage machine may include memory devices such as various hard disk drives, CD, or DVD devices. The logic machine may execute machine-readable instructions via one or more physical information and/or logic processing devices. For example, the logic machine may be configured to execute instructions to perform tasks for a computer program. The logic machine may include one or more processors to execute the machine-readable instructions. The computing system may include a display subsystem to display a graphical user interface (GUI) or any visual element of the methods or processes described above. For example, the display subsystem, storage machine, and logic machine may be integrated such that the above method may be executed while visual elements of the disclosed system and/or method are displayed on a display screen for user consumption. The computing system may include an input subsystem that receives user input. The input subsystem may be configured to connect to and receive input from devices such as a mouse, keyboard or gaming controller. For example, a user input may indicate a request that certain task is to be executed by the computing system, such as requesting the computing system to display any of the above-described information or requesting that the user input updates or modifies existing stored information for processing. A communication subsystem may allow the methods described above to be executed or provided over a computer network. For example, the communication subsystem may be configured to enable the computing system to communicate with a plurality of personal computing devices. The communication subsystem may include wired and/or wireless communication devices to facilitate networked communication. The described methods or processes may be executed, provided, or implemented for a user or one or more computing devices via a computer-program product such as via an application programming interface (API).

As described hereinabove, embodiments of the present invention may comprise an advanced system for automating patient assessment and personalized medical guidance using a large language model (LLM)-based personal health assistant (PHA) chatbot. The system may be configured to dynamically inject patient-specific information into LLM prompts, identify common health patterns among patient users, and/or provide standard medical guidance based on routine screening and vaccine guidelines. Additionally, embodiments of the system may feature multi-language support, customizable virtual physician interfaces, and/or an interactive onboarding process to create a secure healthcare profile within the HealthLynked network. Advantages of the system according to certain embodiments of the present invention may include the following:
Dynamic Patient Interaction: A customizable, avatar-driven interface that leverages LLMs to simulate human conversation, dynamically interpreting patient inputs in real time.
Prompt Injection Technology: A method for automatically incorporating relevant patient data-such as medical history, current medications, and known allergies-into LLM prompts to provide accurate and personalized responses to specific patient inquiries.
Patient Onboarding and Profile Creation: An interactive onboarding process allows patients to speak with the AI-powered ARI to provide their personal healthcare information, including medical history, medications, allergies, and lifestyle details. This information is securely stored in their HealthLynked network profile.
Pattern Recognition Across Patients: A capability to analyze data across multiple patient users to identify common issues related to medical conditions, such as location, medications, genetics, medical history, activities, and environmental exposures. This allows for early detection of public health concerns, trends, and emerging patterns.
Multi-language and Culturally Adaptive Responses: The system can understand and respond in any language, providing culturally sensitive and contextually appropriate medical advice. Patients can select their preferred language, and the system adapts its responses accordingly.
Customizable Virtual Physician Interface: The system offers patients the ability to interact with a virtual physician avatar of their choice, with customizable options for gender (male or female) and ethnicity, enhancing patient comfort and engagement.
Standard Medical Guidance: Integration of routine screening and vaccine guidelines based on age, gender, and medical history, providing patients with up-to-date and personalized health maintenance advice.
Comprehensive Data Management: Integration with existing EMR systems and external data sources, allowing the chatbot to build and maintain an accurate, up-to-date patient profile.
Contextually Aware Recommendations: Utilization of LLM capabilities to analyze patient data and generate personalized health advice, leveraging the model's understanding of context, language nuances, and medical knowledge.
Enhanced Data Security: Incorporates advanced encryption methods and access controls to ensure patient data privacy and compliance with healthcare regulations.

Some of the illustrative aspects of the present invention may be advantageous in solving the problems herein described and other problems not discussed which are discoverable by a skilled artisan.

While the above description contains much specificity, these should not be construed as limitations on the scope of any embodiment, but as exemplifications of the presented embodiments thereof. Many other ramifications and variations are possible within the teachings of the various embodiments. While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best or only mode contemplated for carrying out this invention, but that the invention will include all embodiments falling within the scope of the appended claims. Also, in the drawings and the description, there have been disclosed exemplary embodiments of the invention and, although specific terms may have been employed, they are unless otherwise stated used in a generic and descriptive sense only and not for purposes of limitation, the scope of the invention therefore not being so limited. Moreover, the use of the terms first, second, etc. do not denote any order or importance, but rather the terms first, second, etc. are used to distinguish one element from another. Furthermore, the use of the terms a, an, etc. do not denote a limitation of quantity, but rather denote the presence of at least one of the referenced item.

Thus, the scope of the invention should be determined by the appended claims and their legal equivalents, and not by the examples given.

That which is claimed is:

1. A personal health assistant (PHA) chatbot system configured to perform the operations comprising:
receiving, using an Automated Real-Time Interface (ARI) Subsystem characterized by a large language model (LLM) platform, a patient query of one of an onboarding command type and a recommendation command type;
upon detection, using the ARI Subsystem, of the onboarding command type:
prompting for and receiving, using the ARI Subsystem, a patient identifier, at least one account setup record, and at least one personal healthcare record, and
storing, using a HealthLynked Artificial Intelligence (AI) Subsystem, the at least one personal healthcare record to a secure medical profile associated with the patient identifier and characterized by the at least one account setup record; and
upon detection, using the ARI Subsystem, of the recommendation command type:
prompting for and receiving, using the ARI Subsystem, a patient identifier and a personal medical inquiry record,
creating, using the HealthLynked AI Subsystem, a prompt record based on at least one patient-specific datum of the secure medical profile associated with the patient identifier,
creating, using the ARI Subsystem, a dynamic prompt characterized by the prompt record, and
updating, using the ARI Subsystem, an avatar-based interface based on the dynamic prompt.

2. The personal health assistant (PHA) chatbot system according to claim 1, further comprising a Structured Medical Data store configured to host the secure medical profile.

3. The personal health assistant (PHA) chatbot system according to claim 1, further configured to perform the operation of updating, using the HealthLynked AI Subsystem, the at least one patient-specific datum of the secure medical profile associated with the patient identifier with a patient profile change.

4. The personal health assistant (PHA) chatbot system according to claim 3, wherein the at least one patient-specific datum of the secure medical profile associated with the patient identifier is of a record type selected from the group consisting of medical history, medications, and allergies.

5. The personal health assistant (PHA) chatbot system according to claim 3, further configured to perform the operation of retrieving, using the ARI Subsystem, the patient profile change from an external data source across a network interface.

6. The personal health assistant (PHA) chatbot system according to claim 3, wherein the patient profile change is of an electronic medical record (EMR) type.

7. The personal health assistant (PHA) chatbot system according to claim 1, further configured to perform the operations of:
matching, using the HealthLynked AI Subsystem, a common health issue type selected from the group consisting of location, medications, genetics, medical history, activities, and environmental exposures to the at least one patient-specific datum of the secure medical profile associated with the patient identifier; and
creating, using the HealthLynked AI Subsystem, the prompt record based at least in part on the common health issue type.

8. The personal health assistant (PHA) chatbot system according to claim 1, further configured to perform the operation of creating, using the HealthLynked AI Subsystem, the prompt record based on at least one of a language type, a culture type, an age type, a gender type, and an ethnicity type associated with the patient identifier.

9. The personal health assistant (PHA) chatbot system according to claim 1, further configured to perform the operation of creating, using the HealthLynked AI Subsystem, the prompt record based on one of a medical recommendation and a personalized health advice.

10. The personal health assistant (PHA) chatbot system according to claim 9, wherein the medical recommendation is of one of a routine screening type and a vaccine guideline type.

11. A computer-implemented method of performing personal health assistant (PHA) chatbot operations using a large language model (LLM) platform, the method comprising the steps of:
receiving a patient query of one of an onboarding command type and a recommendation command type;
upon detection of the onboarding command type:
prompting for and receiving a patient identifier, at least one account setup record, and at least one personal healthcare record, and
storing the at least one personal healthcare record to a secure medical profile associated with the patient identifier and characterized by the at least one account setup record; and
upon detection of the recommendation command type:
prompting for and receiving a patient identifier and a personal medical inquiry record,
creating a prompt record based on at least one patient-specific datum of the secure medical profile associated with the patient identifier,
creating a dynamic prompt characterized by the prompt record, and
updating an avatar-based interface based on the dynamic prompt.

12. The computer-implemented method according to claim 11, further comprising the step of updating the at least one patient-specific datum of the secure medical profile associated with the patient identifier with a patient profile change.

13. The computer-implemented method according to claim 12, wherein the at least one patient-specific datum of the secure medical profile associated with the patient identifier is of a record type selected from the group consisting of medical history, medications, and allergies.

14. The computer-implemented method according to claim 12, further comprising the step of retrieving the patient profile change from an external data source across a network interface.

15. The computer-implemented method according to claim 12, wherein the patient profile change is of an electronic medical record (EMR) type.

16. The computer-implemented method according to claim 11, further comprising the steps of:
matching a common health issue type selected from the group consisting of location, medications, genetics, medical history, activities, and environmental exposures to the at least one patient-specific datum of the secure medical profile associated with the patient identifier; and
creating the prompt record based at least in part on the common health issue type.

17. The computer-implemented method according to claim 11, further comprising the step of creating the prompt record based on at least one of a language type, a culture type, an age type, a gender type, and an ethnicity type associated with the patient identifier.

18. The computer-implemented method according to claim 11, further comprising the step of creating the prompt record based on one of a medical recommendation and a personalized health advice.

19. A computer-implemented method of performing personal health assistant (PHA) chatbot operations using a large language model (LLM) platform, the method comprising the steps of:
receiving a patient query of a recommendation command type;
prompting for and receiving a patient identifier and a personal medical inquiry record;
creating a prompt record based on at least one patient-specific datum of a secure medical profile associated with the patient identifier;
creating a dynamic prompt characterized by the prompt record; and
updating an avatar-based interface based on the dynamic prompt.

20. The computer-implemented method according to claim 19, further comprising the steps of:
receiving an onboarding command type;
prompting for and receiving a patient identifier, at least one account setup record, and at least one personal healthcare record; and
storing the at least one personal healthcare record to the secure medical profile associated with the patient identifier and characterized by the at least one account setup record.
